(19) United States
(12) Patent Application Publication
(10) Pub. No.: US 2026/0345590 A1
(43) Pub. Date: Nov. 5, 2026

(54) COMBINATIONS OF ESTROGEN RECEPTOR DEGRADERS AND AKT INHIBITORS

(71) Applicant: Arvinas Operations, Inc., New Haven, CT (US)

(72) Inventors: Jessica Li Fong Teh, Milford, CT (US); Richard Walter Gedrich, Guilford, CT (US)

(21) Appl. No.: 19/487,931

(22) PCT Filed: May 31, 2024
(86) PCT No.: PCT/US2024/032021

§ 371 Date: Nov. 25, 2025

Related U.S. Application Data
(60) Provisional application No. 63/470,515, filed on Jun. 2, 2023.

(51) Int. Cl. A61K 31/496; A61K 31/4155; A61K 31/519; A61P 35/00

(52) U.S. Cl. CPC A61K 31/496; A61K 31/4155; A61K 31/519; A61P 35/00

(57) ABSTRACT

Disclosed herein are methods for treating cancer comprising administering to a subject a daily dose (I) of Compound A, or a pharmaceutically acceptable salt thereof, in combination with an Akt inhibitor.

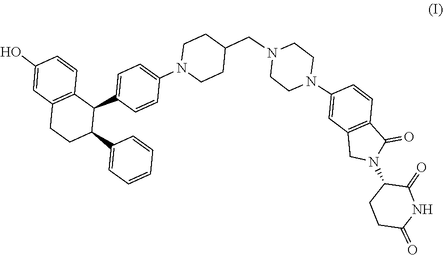 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

### RELATED APPLICATIONS

This application claims priority to, and the benefit of U.S. Provisional Application No. 63/470,515, filed Jun. 2, 2023, the contents of which are incorporated herein by reference in their entirety for all purposes.

### BACKGROUND OF THE INVENTION

Certain bifunctional compounds can target specific cellular proteins for degradation via the ubiquitin-proteasome system. Examples of such proteolysis targeting chimeric compounds (i.e., “PROTAC® protein degraders”) that target the estrogen receptor (ER) for ubiquitination and subsequent degradation are disclosed in International Publication No. WO 2018/102725, which is incorporated herein by reference in its entirety. Such bifunctional molecules exhibit a range of pharmacological activities consistent with the degradation of the ER including, but not limited to, treatment or amelioration of a disease condition such as cancer (e.g., breast cancer, uterine cancer, ovarian cancer, prostate cancer, or endometrial cancer), or endometriosis.

A bifunctional molecule of particular interest is (S)-3-(5-(4-((1-(4-((1R,2S)-6-hydroxy-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione or (3S)-3-[1,3-dihydro-1-oxo-5-[4-[[1-[4-[(1R,2S)-1,2,3,4-tetrahydro-6-hydroxy-2-phenyl-1-naphthalenylphenyl]-4-piperidinyl]methyl]-1-piperazinyl]-2H-isoindol-2-yl]-2,6-piperidinedione (referred to herein as “Compound A” or “Cpd A”), which has the molecular formula of C₄₅H₄₉N₅O₄ and the following structure:

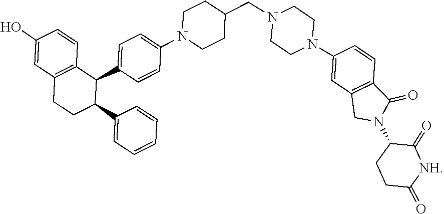 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

Compound A is under development as a PROTAC® protein degrader that targets estrogen receptor (ER) for the potential treatment of breast cancer and has been shown to be a useful modulator of targeted protein ubiquitination and degradation via the ubiquitin-proteasome pathway.

There is a need for appropriate dosage regimens of Compound A as an oral therapy for treating cancers (e.g., breast cancer), to improve its benefit, including safety and efficacy, and convenience to patients while at the same time minimizing adverse events and risks to patients.

### SUMMARY OF THE INVENTION

The present disclosure provides, in part, dosage regimens for administering Compound A, or a pharmaceutically acceptable salt thereof, to a subject in combination therapies, for treating cancer. This summary is provided to introduce a selection of concepts in a simplified form that are further described below in the detailed description. This summary is not intended to identify key features or essential features of the claimed subject matter, nor is it intended to be used in isolation as an aid in determining the scope of the claimed subject matter. Provided herein are methods for treating cancer comprising administering to a subject Compound A having a structure of:

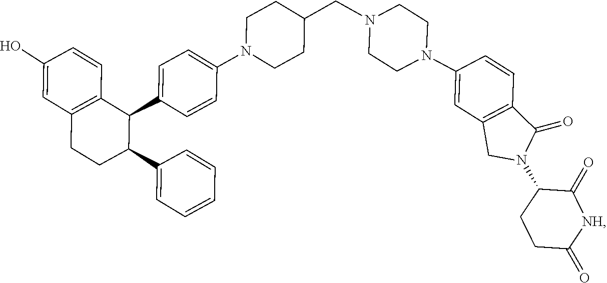 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

or a pharmaceutically acceptable salt thereof, in combination with an Akt inhibitor.

In embodiments, a daily dose of Compound A, or a pharmaceutically acceptable salt thereof, is administered to the subject.

In embodiments, the daily dose of Compound A, or a pharmaceutically acceptable salt thereof, is about 200 mg.

Provided herein are methods for treating cancer comprising administering to a subject Compound A having a structure of:

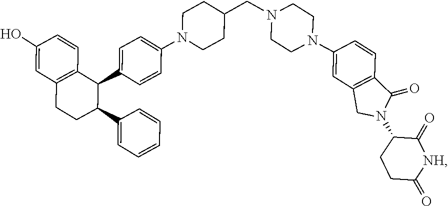 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

in combination with an Akt inhibitor.

In embodiments, a daily dose of Compound A is administered to the subject.

In embodiments, the daily dose of Compound A is about 200 mg.

In embodiments, the daily dose of Compound A is administered once per day (QD).

In embodiments, the daily dose of Compound A is administered orally to the subject.

In embodiments, the Akt inhibitor is capivasertib, afuresertib, ipatasertib, MK-2206, miransertib, or a pharmaceutically acceptable salt thereof. In embodiments, the Akt inhibitor is capivasertib, afuresertib, or a pharmaceutically acceptable salt thereof. In embodiments, the Akt inhibitor is capivasertib, or a pharmaceutically acceptable salt thereof. In embodiments, the Akt inhibitor is afuresertib, or a pharmaceutically acceptable salt thereof.

In embodiments, the subject is in a fed state.

In embodiments, the cancer is breast cancer, lung cancer, colon cancer, brain cancer, head and neck cancer, prostate cancer, stomach cancer, pancreatic cancer, ovarian cancer, melanoma, endocrine cancer, uterine cancer, testicular cancer, or bladder cancer.

In embodiments, the cancer is breast cancer, lung cancer, prostate cancer, pancreatic cancer, or ovarian cancer.

In embodiments, the cancer is breast cancer, lung cancer, or prostate cancer.

In embodiments, the cancer is breast cancer. For example, the breast cancer may be metastatic or locally advanced. Alternatively, the breast cancer may be estrogen receptor positive (ER+) breast cancer (e.g., human epidermal growth factor receptor 2 negative (HER2−)).

In embodiments, the subject is human.

It is to be understood that both the foregoing general description and the following detailed description are exemplary and explanatory only and are not restrictive of the invention, as claimed.

### BRIEF DESCRIPTION OF THE DRAWINGS

FIGS. 1A and 1B show the relative cell growth of MCF7 cells (FIG. 1A) and T47D cells (FIG. 1B) overtime for control (DMSO), capivasertib, compound A, and a combination of capivasertib and compound A. Means−/+standard errors of the mean (SEM) of 3 independent experiments; ***p<0.005; ****p<0.0001.

FIGS. 2A and 2B show the relative cell growth at day 5 of MCF7 cells (FIG. 2A) and T47D cells (FIG. 2B) overtime for control (DMSO), capivasertib, compound A, and a combination of capivasertib and compound A. Means−/+standard errors of the mean (SEM) of 3 independent experiments; ***p<0.005; ****p<0.0001.

FIGS. 3A-3C show the dose-response inhibition of MCF7 cell growth of compound A (FIG. 3A), capivasertib (FIG. 3B), and a combination of compound A and capivasertib (FIG. 3C).

FIGS. 4A-4C show synergy analyses of compound A and capivasertib using the Bliss (Independence) Model (FIG. 4A), Loewe (Additivity) Model (FIG. 4B), and the highest single agent (HSA) model (FIG. 4C).

FIGS. 5A and 5B show the relative cell growth of MCF7 cells (FIG. 5A) and T47D cells (FIG. 5B) overtime for control (DMSO), afuresertib, compound A, and a combination of afuresertib and compound A. Means−/+standard errors of the mean (SEM) of 3 independent experiments; ***p<0.005; ****p<0.0001.

FIGS. 6A and 6B show the relative cell growth at day 5 of MCF7 cells (FIG. 6A) and T47D cells (FIG. 6B) overtime for control (DMSO), afuresertib, compound A, and a combination of afuresertib and compound A. Means−/+standard errors of the mean (SEM) of 3 independent experiments; ***p<0.005; ****p<0.0001.

FIGS. 7A-7C show the dose-response inhibition of MCF7 cell growth of compound A (FIG. 7A), afuresertib (FIG. 7B), and a combination of compound A and afuresertib (FIG. 7C).

FIGS. 8A-8C show synergy analyses of compound A and afuresertib using the Bliss (Independence) Model (FIG. 8A), Loewe (Additivity) Model (FIG. 8B), and the highest single agent (HSA) model (FIG. 8C).

### DETAILED DESCRIPTION OF THE INVENTION

The present invention may be understood more readily by reference to the following detailed description of the embodiments of the invention and the Examples included herein. It is to be also understood that the terminology used herein is for the purpose of describing specific embodiments only and is not intended to be limiting.

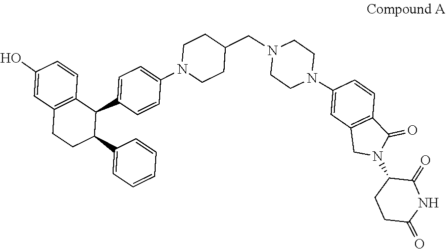 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

is under development as a PROTAC® protein degrader that targets estrogen receptor (ER) for the potential treatment of breast cancer and has been shown to be a useful modulator of targeted protein ubiquitination and degradation via the ubiquitin-proteasome pathway.

Compound A and pharmaceutically acceptable salts thereof are disclosed in International Publication No. WO 2018/102725 and U.S. Pat. Nos. 10,647,698, 10,899,742 and 11,104,666; International Publication No. WO 2021/041348; U.S. Ser. No. 17/472,847; U.S. Ser. No. 17/548,842; and U.S. Ser. No. 17/873,748. The contents of each of the foregoing references are incorporated herein by reference in their entirety.

Protein kinase B (PKB), also known as Akt, is the collective name of a set of three serine/threonine-specific protein kinases that play key roles in multiple cellular processes such as glucose metabolism, apoptosis, cell proliferation, transcription, and cell migration. There are three different genes that encode isoforms of Protein kinase B. These three genes are referred to as AKT1, AKT2, and AKT3 and encode the RAC alpha, beta, and gamma serine/threonine protein kinases respectively. The terms PKB and Akt may refer to the products of all three genes collectively, but sometimes are used to refer to PKB alpha and Akt1 alone. Akt1 is involved in cellular survival pathways, by inhibiting apoptotic processes. Akt1 is also able to induce protein synthesis pathways and is therefore a key signaling protein in the cellular pathways that lead to skeletal muscle hypertrophy and general tissue growth. A mouse model with complete deletion of the Akt1 gene manifests growth retardation and increased spontaneous apoptosis in tissues such as testes and thymus (Chen W S, Xu P Z, Gottlob K, Chen M L, Sokol K, Shiyanova T, et al. (2001). Growth retardation and increased apoptosis in mice with homozygous disruption of the Akt1 gene. Genes & Development. 15 (17): 2203-08). Since it can block apoptosis and thereby promote cell survival, Akt1 has been implicated as a major factor in many types of cancer. Akt1 was originally identified as the oncogene in the transforming retrovirus, AKT8 (Staal S P, Hartley J W, Rowe W P (1977). Isolation of transforming murine leukemia viruses from mice with a high incidence of spontaneous lymphoma. Proceedings of the National Academy of Sciences of the United States of America. 74 (7): 3065-67).

Analysis of Akt levels in human tumors showed that Akt2 is overexpressed in a significant number of ovarian (J. Q. Cheung et al. Proc. Natl. Acad. Sci. U.S.A. 89:9267-9271 (1992)) and pancreatic cancers (J. Q. Cheung et al. Proc. Natl. Acad. Sci. U.S.A. 93:3636-3641 (1996)). Similarly, Akt3 was found to be overexpressed in breast and prostate cancer cell lines (Nakatani et al. J. Biol. Chem. 274:21528-21532 (1999). It was demonstrated that AKT2 was over-expressed in 12% of ovarian carcinomas and that amplification of AKT was especially frequent in 50% of undifferentiated tumors, suggestion that AKT may also be associated with tumor aggressiveness (Bellacosa, et al., Int. J. Cancer, 64, pp. 280-285, 1995). Increased Akt1 kinase activity has been reported in breast, ovarian and prostate cancers (Sun et al. Am. J. Pathol. 159:431-7 (2001)).

The tumor suppressor PTEN, a protein and lipid phosphatase that specifically removes the 3′ phosphate of PtdIns(3,4,5)P₃, is a negative regulator of the PI3K/Akt pathway (Li et al. Science 275:1943-1947 (1997), Stambolic et al. Cell 95:29-39 (1998), Sun et al. Proc. Natl. Acad. Sci. U.S.A. 96:6199-204 (1999)). Germline mutations of PTEN are responsible for human cancer syndromes such as Cowden disease (Liaw et al. Nature Genetics 16:64-67 (1997)). PTEN is deleted in a large percentage of human tumors and tumor cell lines without functional PTEN show elevated levels of activated Akt (Li et al. supra, Guldberg et al. Cancer Research 57:3660-63 (1997), Risinger et al. Cancer Research 57:4736-38 (1997)). These observations demonstrate that the Pl3K/Akt pathway plays important roles for regulating cell survival or apoptosis in tumorigenesis.

Small molecule inhibitors of AKT are useful in the treatment of tumors, especially those with activated AKT (e.g., PTEN null tumors and tumors with ras mutations). PTEN is a critical negative regulator of AKT and its function is lost in many cancers, including breast and prostate carcinomas, glioblastomas, and several cancer syndromes including Bannayan-Zonana syndrome (Maehama, T. et al. Annual Review of Biochemistry, 70:247 (2001)), Cowden disease (Parsons, R.; Simpson, L. Methods in Molecular Biology (Totowa, N.J., United States), 222 (Tumor Suppressor Genes, Volume 1): 147 (2003)), and Lhermitte-Duclos disease (Backman, S. et al. Current Opinion in Neurobiology, 12 (5): 516 (2002)).

Capivasertib, 4-amino-N-[(1S)-1-(4-chlorophenyl)-3-hydroxypropyl]-1-(7H-pyrrolo[2,3-d]pyrimidin-4-yl)piperidine-4-carboxamide, (also referred to as “AZD-5363”) is an orally bioavailable serine/threonine protein kinase AKT (protein kinase B), having the structure:

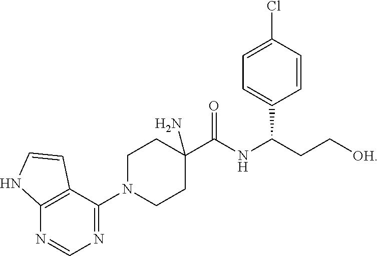 [SMILES: NC1(C(=O)N[C@@H](CCO)c2ccc(Cl)cc2)CCN(c2ncnc3[nH]ccc23)CC1]

Capivasertib, or a pharmaceutically acceptable salt thereof, is disclosed in International Publication No. 2009/047563, U.S. Pat. Nos. 8,101,623, 10,059,714, 10,654,855, 11,236,095, and U.S. Patent Publication No. 2022/022011. The contents of each of the foregoing references are incorporated herein by reference in their entirety. Capivasertib binds to and inhibits all AKT isoforms. Inhibition of AKT prevents the phosphorylation of AKT substrates that mediate cellular processes, such as cell division, apoptosis, and glucose and fatty acid metabolism.

Afuresertib, N-[(2S)-1-amino-3-(3-fluorophenyl)propan-2-yl]-5-chloro-4-(4-chloro-2-methyl-3-pyrazolyl)-2-thiophenecarboxamide, (also referred to as “GSK-2110183” or “GSK2110183C”) is an orally bioavailable serine/threonine protein kinase AKT (protein kinase B), having the structure:

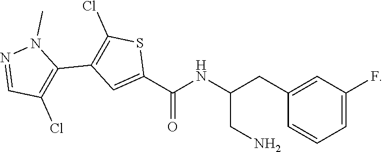 [SMILES: Cn1ncc(Cl)c1-c1cc(C(=O)NC(CN)Cc2cccc(F)c2)sc1Cl]

Afuresertib, or a pharmaceutically acceptable salt thereof, is disclosed in International Publication Nos. 2008/098104 and U.S. Pat. Nos. 8,410,158 and 8,946,278. Afuresertib is a reversible, ATP-competitive, low-nanomolar, pan-AKT kinase inhibitor.

### DEFINITIONS

Unless otherwise defined herein, scientific, and technical terms used in connection with the present invention have the meanings that are commonly understood by those of ordinary skill in the art.

The invention described herein suitably may be practiced in the absence of any element(s) not specifically disclosed herein.

As used herein, the singular form “a,” “an,” and “the” include plural references unless indicated otherwise. For example, “a” substituent includes one or more substituents.

As used herein, the term “about” when used to modify a numerically defined parameter (e.g., the dose of a compound) means that the parameter may vary by as much as 10% below or above the stated numerical value for that parameter. For example, a dose of about 5 mg means 5 mg±10%, i.e., it may vary from 4.5 mg to 5.5 mg.

As used herein, terms, including, but not limited to, “agent,” “composition,” “compound,” “drug,” and “therapeutic agent” may be used interchangeably to refer to compounds included in the methods and uses of the present disclosure.

As used herein, the terms, “subject,” “participant,” and “patient,” are used interchangeably, to refer to any animal, including mammals. Mammals according to the disclosure include canine, feline, bovine, caprine, equine, ovine, porcine, rodents, lagomorphs, primates, humans, and the like, and encompass mammals in utero. In embodiments, humans are suitable subjects. Human subjects may be of any gender and at any stage of development.

Compound A is a Biopharmaceutics Classification System Class IV compound (low solubility/low permeability). Compound A can interconvert to its epimer, Compound C:

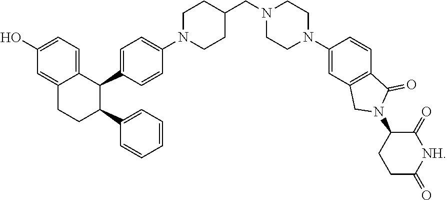 [SMILES: O=C1CC[C@@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

Without wishing to be bound by theory, preclinical data indicates that the exposure of Compound C is limited compared to Compound A (<26%). Evidence indicates that Compound C does not degrade the ER; however, Compound C shows similar antagonism of ERdependent transcription compared to Compound A.

Protein kinase B (PKB), also known as Akt, is the collective name of a set of three serine/threonine-specific protein kinases that play key roles in multiple cellular processes such as glucose metabolism, apoptosis, cell proliferation, transcription, and cell migration. Akt inhibitors include pan-Akt inhibitors that target a broad spectrum of Akts or selective Akt inhibitors that target specific Akt(s).

In embodiments, a pan-Akt inhibitor of the present invention includes capivasertib, 4-amino-N-[(1S)-1-(4-chlorophenyl)-3-hydroxypropyl]-1-(7H-pyrrolo[2,3-d]pyrimidin-4-yl)piperidine-4-carboxamide, (also referred to as “AZD-5363”), having the structure:

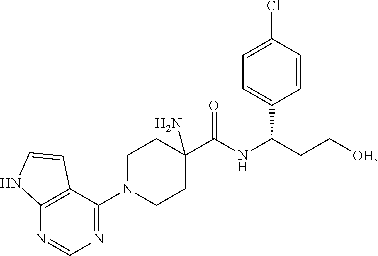 [SMILES: NC1(C(=O)N[C@@H](CCO)c2ccc(Cl)cc2)CCN(c2ncnc3[nH]ccc23)CC1]

or a pharmaceutically acceptable salt thereof.

In embodiments, a pan-Akt inhibitor of the present invention includes afuresertib, N-[(2S)-1-amino-3-(3-fluorophenyl)propan-2-yl]-5-chloro-4-(4-chloro-2-methyl-3-pyrazolyl)-2-thiophenecarboxamide, (also referred to as “GSK-2110183” or “GSK2110183C”), having the structure:

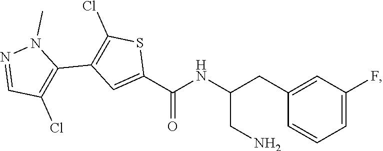 [SMILES: Cn1ncc(Cl)c1-c1cc(C(=O)NC(CN)Cc2cccc(F)c2)sc1Cl]

or a pharmaceutically acceptable salt thereof.

Other embodiments relate to the pharmaceutically acceptable salts of the compounds described herein. Pharmaceutically acceptable salts of the compounds described herein include the acid addition and base addition salts thereof.

Other embodiments also relate to the pharmaceutically acceptable acid addition salts of the compounds described herein. Suitable acid addition salts are formed from acids which form non-toxic salts. Non-limiting examples of suitable acid addition salts, i.e., salts containing pharmacologically acceptable anions, include, but are not limited to, the acetate, acid citrate, adipate, aspartate, benzoate, besylate, bicarbonate/carbonate, bisulphate/sulphate, bitartrate, borate, camsylate, citrate, cyclamate, edisylate, esylate, ethanesulfonate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methanesulfonate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, p-toluenesulfonate, tosylate, trifluoroacetate and xinofoate salts.

Additional embodiments relate to base addition salts of the compounds described herein. Suitable base addition salts are formed from bases that form non-toxic salts. Non-limiting examples of suitable base salts include the aluminum, arginine, benzathine, calcium, choline, diethylamine, diolamine, glycine, lysine, magnesium, meglumine, olamine, potassium, sodium, tromethamine, and zinc salts.

The compounds described herein that are basic in nature can form a wide variety of salts with various inorganic and organic acids. The acids that may be used to prepare pharmaceutically acceptable acid addition salts of such basic compounds described herein are those that form non-toxic acid addition salts, e.g., salts containing pharmacologically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate [i.e., 1,1′-methylene-bis-(2-hydroxy-3-naphthoate)] salts. The compounds described herein that include a basic moiety, such as an amino group, may form pharmaceutically acceptable salts with various amino acids, in addition to the acids mentioned above.

The chemical bases that may be used as reagents to prepare pharmaceutically acceptable base salts of those compounds of the compounds described herein that are acidic in nature are those that form non-toxic base salts with such compounds. Such non-toxic base salts include, but are not limited to, those derived from such pharmacologically acceptable cations such as alkali metal cations (e.g., potassium and sodium) and alkaline earth metal cations (e.g., calcium and magnesium), ammonium or water-soluble amine addition salts such as N-methylglucamine-(meglumine), and the lower alkanolammonium and other base salts of pharmaceutically acceptable organic amines.

Hemisalts of acids and bases may also be formed, for example, hemisulphate, and hemicalcium salts.

For a review on suitable salts, see Handbook of Pharmaceutical Salts: Properties, Selection, and Use by Stahl and Wermuth (Wiley-VCH, 2002). Methods for making pharmaceutically acceptable salts of compounds described herein are known to one of skill in the art.

### Combinations

Provided herein are methods for treating cancer comprising administering to a subject Compound A:

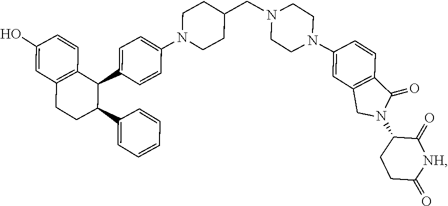 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

or a pharmaceutically acceptable salt thereof, in combination with an Akt inhibitor.

In embodiments, the subject is administered a daily dose of Compound A, or a pharmaceutically acceptable salt thereof.

In embodiments, the daily dose of Compound A, or a pharmaceutically acceptable salt thereof, is administered once per day (QD).

In embodiments, the daily dose of Compound A, or a pharmaceutically acceptable salt thereof, is administered orally to the subject.

In embodiments, the subject is in a fed state.

In embodiments, the daily dose of Compound A, or a pharmaceutically acceptable salt thereof, is about 200 mg.

In embodiments, the daily dose of Compound A, or a pharmaceutically acceptable salt thereof, is 200 mg.

In embodiments, the daily dose of Compound A, or a pharmaceutically acceptable salt thereof, is about 100 mg.

In embodiments, the daily dose of Compound A, or a pharmaceutically acceptable salt thereof, is 100 mg.

In embodiments, Compound A, or a pharmaceutically acceptable salt thereof, is administered as a free base.

Provided herein are methods for treating cancer comprising administering to a subject Compound A:

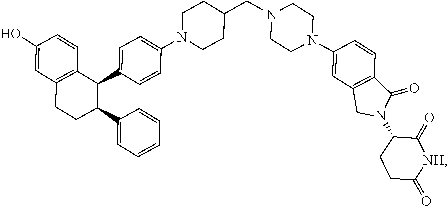 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

in combination with an Akt inhibitor.

In embodiments, the subject is administered a daily dose of Compound A.

In embodiments, the daily dose of Compound A is administered once per day (QD).

In embodiments, the daily dose of Compound A is administered orally to the subject.

In embodiments, the daily dose of Compound A is about 200 mg.

In embodiments, the daily dose of Compound A is 200 mg.

In embodiments, the daily dose of Compound A is about 100 mg.

In embodiments, the daily dose of Compound A is 100 mg.

In embodiments, Compound A is administered as a free base.

In embodiments, the Akt inhibitor is capivasertib, afuresertib, ipatasertib, MK-2206, miransertib, or a pharmaceutically acceptable salt thereof. In embodiments, the Akt inhibitor is capivasertib, afuresertib, or a pharmaceutically acceptable salt thereof. In embodiments, the Akt inhibitor is capivasertib, or a pharmaceutically acceptable salt thereof. In embodiments, the Akt inhibitor is afuresertib, or a pharmaceutically acceptable salt thereof.

In embodiments, the Akt inhibitor is capivasertib, afuresertib, ipatasertib, MK-2206, or miransertib. In embodiments, the Akt inhibitor is capivasertib or afuresertib. In embodiments, the Akt inhibitor is capivasertib. In embodiments, the Akt inhibitor is afuresertib.

In embodiments, Compound A, or a pharmaceutically acceptable salt thereof, may be administered daily in 28-day cycles in combination with the Akt inhibitor. In certain embodiments, capivasertib, or a pharmaceutically acceptable salt thereof, is administered orally once a day for 21 days followed by 7 days off treatment for each 28-day cycle. For example, capivasertib may be administered orally once a day for 21 days followed by 7 days off treatment for each 28-day cycle. In certain embodiments, capivasertib, or a pharmaceutically acceptable salt thereof, is administration orally twice daily for 4 consecutive days followed by 3 days off treatment each week to comprise a complete cycle of 28-days.

In embodiments, Compound A, or a pharmaceutically acceptable salt thereof, may be administered daily in 28-day cycles in combination with the Akt inhibitor. In certain embodiments, afuresertib, or a pharmaceutically acceptable salt thereof, is administered orally once a day for 21 days followed by 7 days off treatment for each 28-day cycle. For example, afuresertib may be administered orally once a day for 21 days followed by 7 days off treatment for each 28-day cycle.

In embodiments, the cancer is breast cancer, lung cancer, colon cancer, brain cancer, head and neck cancer, prostate cancer, stomach cancer, pancreatic cancer, ovarian cancer, melanoma, endocrine cancer, uterine cancer, testicular cancer, or bladder cancer.

In embodiments, the cancer is breast cancer, lung cancer, prostate cancer, pancreatic cancer, or ovarian cancer.

In embodiments, the cancer is breast cancer, lung cancer, or prostate cancer.

In embodiments, the cancer is breast cancer.

In embodiments, the breast cancer is metastatic or locally advanced.

In embodiments, the breast cancer is estrogen receptor positive (ER+) breast cancer.

In embodiments, the estrogen receptor positive (ER+) breast cancer is human epidermal growth factor receptor 2 negative (HER2−).

In embodiments, the subject is human.

Also disclosed herein is Compound A, or a pharmaceutically acceptable salt thereof, for use according to any one of foregoing embodiments.

Also disclosed herein is Compound A for use according to any one of foregoing embodiments.

Also disclosed herein are uses of Compound A, or a pharmaceutically acceptable salt thereof, in the manufacture of a medicament according to any one of foregoing embodiments.

Also disclosed herein are uses of Compound A in the manufacture of a medicament according to any one of foregoing embodiments.

Each of the embodiments described herein may be combined with any other embodiment(s) described herein not inconsistent with the embodiment(s) with which it is combined.

### Administration and Dosing

The terms “treat” and “treating” a cancer or a cancer-associated disease, as used herein, mean to administer a combination therapy according to the present disclosure to a subject, participant or patient having a cancer, or diagnosed with a cancer, to achieve at least one positive therapeutic effect, such as, for example, reduced number of cancer cells, reduced tumor size, reduced rate of cancer cell infiltration into peripheral organs, or reduced rate of tumor metastasis or tumor growth, reversing, alleviating, inhibiting the progress of, or preventing the disorder or condition to which such term applies, or one or more symptoms of such disorder or condition. The terms “treatment” and “therapy,” as used herein, unless otherwise indicated, refer to the act of treating as “treating” is defined immediately above. For the purposes of this disclosure, beneficial or desired clinical results include, but are not limited to, one or more of the following: reducing the proliferation of (or destroying) neoplastic or cancerous cell; inhibiting metastasis or neoplastic cells; shrinking or decreasing the size of tumor; remission of the cancer; decreasing symptoms resulting from the cancer; increasing the quality of life of those suffering from the cancer; decreasing the dose of other medications required to treat the cancer; delaying the progression the cancer; curing the cancer; overcoming one or more resistance mechanisms of the cancer; and/or prolonging survival of patients the cancer. Positive therapeutic effects in cancer can be measured in a number of ways (see, for example, W. A. Weber, J. Nucl. Med. (2009) 50:1S-10S).

“Fed condition” or “fed state” as used to describe a subject herein, means that the subject has eaten less than 4 hours before a time point of interest, such as the time of administering Compound A. In embodiments, a subject in the fed state has not eaten for at most any of 4, 3, 2, 1, or 0.5 hours prior to administration of Compound A.

An “amount” for use and for treating a subject refers to an amount that provides, in single or multiple doses, in combination with one or more other agents, a detectable response of any duration of time (transient, medium, or long term), a desired outcome in or an objective or subjective benefit to a subject of any measurable or detectable degree or for any duration of time (e.g., for hours, days, months, years, in remission or cured). Such amounts typically are effective to ameliorate a disease, or one, multiple or all adverse effects/symptoms, consequences, or complications of the disease, to a measurable extent, although reducing or inhibiting a progression or worsening of the disease, or providing stability (i.e., not worsening) state of the disease, is considered a satisfactory outcome. The term “therapeutically effective amount” also means an amount of an agent in combination with one or more other agents, effective for producing a desired therapeutic effect upon administration to a subject, for example, to stem the growth, or result in the shrinkage, of a cancerous tumor. In reference to the treatment of cancer, a therapeutically effective amount refers to that amount that has the effect of (1) reducing the size of the tumor, (2) inhibiting (that is, slowing to some extent, preferably stopping) tumor metastasis emergence, (3) inhibiting to some extent (that is, slowing to some extent, preferably stopping) tumor growth or tumor invasiveness, and/or (4) relieving to some extent (or, preferably, eliminating) one or more signs or symptoms associated with the cancer. Therapeutic or pharmacological effectiveness of the doses and administration regimens also may be characterized as the ability to induce, enhance, maintain, or prolong disease control and/or overall survival in patients with these specific tumors, which may be measured as prolongation of the time before disease progression.

As used herein, “ameliorate” refers to any reduction in the extent, severity, frequency, and/or likelihood of a symptom or clinical sign characteristic of a particular disease. “Symptom” refers to any subjective evidence of disease or of a subject's condition.

As used herein, “daily dose” refers to a dose that is administered each day, such as an amount of Compound A that is administered each day.

The daily dose or daily dosage of Compound A, or a pharmaceutically acceptable salt thereof, is the free base equivalent of Compound A. Dosage amounts provided herein refer to the dose of the free base form of Compound A, or are calculated as the free base equivalent of an administered Compound A salt form. For example, a dosage or amount of Compound A, such as about 100 mg or about 200 mg refers to the free base equivalent.

Embodiments of the present invention provide a dose, dosage, and dosing regimen comprising administering to a subject an amount, or an effective amount of Compound A, or a pharmaceutically acceptable salt thereof. The amount, or the therapeutically effective amount, can be a daily dose of about 200 mg. In another embodiment, a daily dose is 200 mg. The amount, or the therapeutically effective amount, can be a daily dose of about 100 mg. In another embodiment, a daily dose is 100 mg.

In embodiments, the daily dose of Compound A, or a pharmaceutically acceptable salt thereof, is administered once per day (QD).

The compounds disclosed herein may be administered orally. Oral administration may involve swallowing, so that the compound enters the gastrointestinal tract, or buccal or sublingual administration may be employed by which the compound enters the bloodstream directly from the mouth.

In embodiments, the daily dose of Compound A, or a pharmaceutically acceptable salt thereof, is administered orally.

Compound A, or a pharmaceutically acceptable salt thereof, may be present in a pharmaceutical composition, which includes a pharmaceutically acceptable excipient. A “pharmaceutically acceptable excipient” refers to a component that may be included in the compositions described herein, is physiologically suitable for pharmaceutical use, and causes no significant adverse effects nor therapeutic effects to a subject. The term “excipient” is used herein to describe any ingredient other than the compound(s) of the invention. The choice of excipient will to a large extent depend on factors such as the mode of administration, the effect of the excipient on solubility and stability, and the nature of the dosage form.

The compounds of the methods, uses, or combinations of the present invention may be formulated prior to administration. The formulation preferably will be adapted to the particular mode of administration. These compounds may be formulated with pharmaceutically acceptable excipients as known in the art and administered in a wide variety of dosage forms as known in the art. Dosage unit forms or pharmaceutical compositions suitable for oral administration include, but are not limited to tablets, capsules, such as gelatin capsules, pills, powders, granules, aqueous, and nonaqueous oral solutions and suspensions, packaged in containers adapted for subdivision into individual doses.

In embodiments, capivasertib, or a pharmaceutically acceptable salt thereof, is administered in the range of from about 100 mg to about 1000 mg once daily. In embodiments, capivasertib, or a pharmaceutically acceptable salt thereof, is administered at a dosage from about 150 mg to about 900 mg once daily. In embodiments, capivasertib, or a pharmaceutically acceptable salt thereof, is administered at a dosage from about 200 mg to about 850 mg once daily. In embodiments, capivasertib, or a pharmaceutically acceptable salt thereof, is administered at a dosage from about 250 mg to about 800 mg once daily. In embodiments, capivasertib, or a pharmaceutically acceptable salt thereof, is administered at a dosage from about 300 mg to about 750 mg once daily. In embodiments, capivasertib, or a pharmaceutically acceptable salt thereof, is administered at a dosage from about 350 mg to about 700 mg once daily. In embodiments, capivasertib, or a pharmaceutically acceptable salt thereof, is administered at a dosage from about 400 mg to about 650 mg once daily.

In embodiments, capivasertib, or a pharmaceutically acceptable salt thereof, is administered to the subject twice daily (BID). In embodiments, capivasertib, or a pharmaceutically acceptable salt thereof, is administered at a dosage from about 50 mg to about 900 mg twice daily. In embodiments, capivasertib, or a pharmaceutically acceptable salt thereof, is administered at a dosage from about 100 mg to about 875 mg twice daily. In embodiments, capivasertib, or a pharmaceutically acceptable salt thereof, is administered at a dosage from about 200 mg to about 850 mg twice daily. In embodiments, capivasertib, or a pharmaceutically acceptable salt thereof, is administered at a dosage from about 250 mg to about 825 mg twice daily. In embodiments, capivasertib, or a pharmaceutically acceptable salt thereof, is administered at a dosage from about 150 mg to about 250 mg twice daily. In embodiments, capivasertib, or a pharmaceutically acceptable salt thereof, is administered at a dosage from about 250 mg to about 350 mg twice daily. In embodiments, capivasertib, or a pharmaceutically acceptable salt thereof, is administered at a dosage from about 350 mg to about 450 mg twice daily. In embodiments, capivasertib, or a pharmaceutically acceptable salt thereof, is administered at a dosage from about 450 mg to about 550 mg twice daily. In embodiments, capivasertib, or a pharmaceutically acceptable salt thereof, is administered at a dosage from about 550 mg to about 650 mg twice daily. In embodiments, capivasertib, or a pharmaceutically acceptable salt thereof, is administered at a dosage from about 650 mg to about 750 mg twice daily. In embodiments, capivasertib, or a pharmaceutically acceptable salt thereof, is administered at a dosage from about 750 mg to about 850 mg twice daily. In embodiments, capivasertib, or a pharmaceutically acceptable salt thereof, is administered at a dosage of about 160 mg twice daily. In embodiments, capivasertib, or a pharmaceutically acceptable salt thereof, is administered at a dosage of about 200 mg twice daily. In embodiments, capivasertib, or a pharmaceutically acceptable salt thereof, is administered at a dosage of about 240 mg twice daily. In embodiments, capivasertib, or a pharmaceutically acceptable salt thereof, is administered at a dosage of about 280 mg twice daily. In embodiments, capivasertib, or a pharmaceutically acceptable salt thereof, is administered at a dosage of about 320 mg twice daily. In embodiments, capivasertib, or a pharmaceutically acceptable salt thereof, is administered at a dosage of about 360 mg twice daily. In embodiments, capivasertib, or a pharmaceutically acceptable salt thereof, is administered at a dosage of about 400 mg twice daily. In embodiments, capivasertib, or a pharmaceutically acceptable salt thereof, is administered at a dosage of about 440 mg twice daily. In embodiments, capivasertib, or a pharmaceutically acceptable salt thereof, is administered at a dosage of about 480 mg twice daily. In embodiments, capivasertib, or a pharmaceutically acceptable salt thereof, is administered at a dosage of about 520 mg twice daily. In embodiments, capivasertib, or a pharmaceutically acceptable salt thereof, is administered at a dosage of about 560 mg twice daily. In embodiments, capivasertib, or a pharmaceutically acceptable salt thereof, is administered at a dosage of about 600 mg twice daily. In embodiments, capivasertib, or a pharmaceutically acceptable salt thereof, is administered at a dosage of about 640 mg twice daily. In embodiments, capivasertib, or a pharmaceutically acceptable salt thereof, is administered at a dosage of about 680 mg twice daily. In embodiments, capivasertib, or a pharmaceutically acceptable salt thereof, is administered at a dosage of about 720 mg twice daily. In embodiments, capivasertib, or a pharmaceutically acceptable salt thereof, is administered at a dosage of about 760 mg twice daily. In embodiments, capivasertib, or a pharmaceutically acceptable salt thereof, is administered at a dosage of about 800 mg twice daily.

In embodiments, capivasertib, or a pharmaceutically acceptable salt thereof, is administered once daily (QD) under a continuous dosing schedule. In embodiments, capivasertib, or a pharmaceutically acceptable salt thereof, is administered once daily under a continuous dosing schedule at a dosage from about 100 mg to about 900 mg. In embodiments, capivasertib, or a pharmaceutically acceptable salt thereof, is administered once daily under a continuous dosing schedule at a dosage from about 150 mg to about 875 mg. In embodiments, capivasertib, or a pharmaceutically acceptable salt thereof, is administered once daily under a continuous dosing schedule at a dosage from about 175 mg to about 850 mg. In embodiments, capivasertib, or a pharmaceutically acceptable salt thereof, is administered once daily under a continuous dosing schedule at a dosage from about 200 mg to about 825 mg. In embodiments, capivasertib, or a pharmaceutically acceptable salt thereof, is administered once daily under a continuous dosing schedule at a dosage from about 225 mg to about 800 mg. In embodiments, capivasertib, or a pharmaceutically acceptable salt thereof, is administered once daily under a continuous dosing schedule at a dosage from about 250 mg to about 750 mg. In embodiments, capivasertib, or a pharmaceutically acceptable salt thereof, is administered once daily under a continuous dosing schedule at a dosage from about 275 mg to about 700 mg. In embodiments, capivasertib, or a pharmaceutically acceptable salt thereof, is administered once daily under a continuous dosing schedule at a dosage from about 300 mg to about 650 mg.

In embodiments, capivasertib, or a pharmaceutically acceptable salt thereof, is administered twice daily (BID) under a continuous dosing schedule. In embodiments, capivasertib, or a pharmaceutically acceptable salt thereof, is administered under a continuous dosing schedule at a dosage from about 100 mg to about 800 mg twice daily. In embodiments, capivasertib, or a pharmaceutically acceptable salt thereof, is administered under a continuous dosing schedule at a dosage from about 150 mg to about 750 mg twice daily. In embodiments, capivasertib, or a pharmaceutically acceptable salt thereof, is administered under a continuous dosing schedule at a dosage from about 200 mg to about 700 mg twice daily. In embodiments, capivasertib, or a pharmaceutically acceptable salt thereof, is administered under a continuous dosing schedule at a dosage from about 225 mg to about 650 mg twice daily. In embodiments, capivasertib, or a pharmaceutically acceptable salt thereof, is administered under a continuous dosing schedule at a dosage from about 250 mg to about 650 mg twice daily. In embodiments, capivasertib, or a pharmaceutically acceptable salt thereof, is administered under a continuous dosing schedule at a dosage from about 300 mg to about 600 mg twice daily. In embodiments, capivasertib, or a pharmaceutically acceptable salt thereof, is administered under a continuous dosing schedule at a dosage from about 200 mg to about 300 mg twice daily. In embodiments, capivasertib, or a pharmaceutically acceptable salt thereof, is administered under a continuous dosing schedule at a dosage from about 300 mg to about 400 mg twice daily. In embodiments, capivasertib, or a pharmaceutically acceptable salt thereof, is administered under a continuous dosing schedule at a dosage from about 400 mg to about 500 mg twice daily. In embodiments, capivasertib, or a pharmaceutically acceptable salt thereof, is administered under a continuous dosing schedule at a dosage from about 500 mg to about 600 mg twice daily. In embodiments, capivasertib, or a pharmaceutically acceptable salt thereof, is administered under a continuous dosing schedule at a dosage from about 600 mg to about 700 mg twice daily. In embodiments, capivasertib, or a pharmaceutically acceptable salt thereof, is administered under a continuous dosing schedule at a dosage from about 700 mg to about 800 mg twice daily. In embodiments, capivasertib, or a pharmaceutically acceptable salt thereof, is administered under a continuous dosing schedule at a dosage of about 160 mg twice daily. In embodiments, capivasertib, or a pharmaceutically acceptable salt thereof, is administered under a continuous dosing schedule at a dosage of about 200 mg twice daily. In embodiments, capivasertib, or a pharmaceutically acceptable salt thereof, is administered under a continuous dosing schedule at a dosage of about 240 mg twice daily. In embodiments, capivasertib, or a pharmaceutically acceptable salt thereof, is administered under a continuous dosing schedule at a dosage of about 280 mg twice daily. In embodiments, capivasertib, or a pharmaceutically acceptable salt thereof, is administered under a continuous dosing schedule at a dosage of about 320 mg twice daily. In embodiments, capivasertib, or a pharmaceutically acceptable salt thereof, is administered under a continuous dosing schedule at a dosage of about 360 mg twice daily. In embodiments, capivasertib, or a pharmaceutically acceptable salt thereof, is administered under a continuous dosing schedule at a dosage of about 400 mg twice daily. In embodiments, capivasertib, or a pharmaceutically acceptable salt thereof, is administered under a continuous dosing schedule at a dosage of about 440 mg twice daily. In another aspect, capivasertib, or a pharmaceutically acceptable salt thereof, is administered under a continuous dosing schedule at a dosage of about 480 mg twice daily. In embodiments, capivasertib, or a pharmaceutically acceptable salt thereof, is administered under a continuous dosing schedule at a dosage of about 520 mg twice daily. In embodiments, capivasertib, or a pharmaceutically acceptable salt thereof, is administered under a continuous dosing schedule at a dosage of about 580 mg twice daily. In embodiments, capivasertib, or a pharmaceutically acceptable salt thereof, is administered under a continuous dosing schedule at a dosage of about 600 mg twice daily. In embodiments, capivasertib, or a pharmaceutically acceptable salt thereof, is administered under a continuous dosing schedule at a dosage of about 640 mg twice daily. In embodiments, capivasertib, or a pharmaceutically acceptable salt thereof, is administered under a continuous dosing schedule at a dosage of about 680 mg twice daily. In embodiments, capivasertib, or a pharmaceutically acceptable salt thereof, is administered under a continuous dosing schedule at a dosage of about 720 mg twice daily. In embodiments, capivasertib, or a pharmaceutically acceptable salt thereof, is administered under a continuous dosing schedule at a dosage of about 760 mg twice daily. In embodiments, capivasertib, or a pharmaceutically acceptable salt thereof, is administered under a continuous dosing schedule at a dosage of about 800 mg twice daily.

In embodiments, capivasertib, or a pharmaceutically acceptable salt thereof, is administered once daily (QD) under an intermittent dosing schedule. In embodiments, capivasertib, or a pharmaceutically acceptable salt thereof, is administered once daily under an intermittent dosing schedule at a dosage from about 100 mg to about 900 mg. In embodiments, capivasertib, or a pharmaceutically acceptable salt thereof, is administered once daily under an intermittent dosing schedule at a dosage from about 150 mg to about 850 mg. In embodiments, capivasertib, or a pharmaceutically acceptable salt thereof, is administered once daily under an intermittent dosing schedule at a dosage from about 175 mg to about 800 mg. In embodiments, capivasertib, or a pharmaceutically acceptable salt thereof, is administered once daily under an intermittent dosing schedule at a dosage from about 200 mg to about 750 mg. In embodiments, capivasertib, or a pharmaceutically acceptable salt thereof, is administered once daily under an intermittent dosing schedule at a dosage from about 225 mg to about 725 mg. In embodiments, capivasertib, or a pharmaceutically acceptable salt thereof, is administered once daily under an intermittent dosing schedule at a dosage from about 250 mg to about 700 mg. In embodiments, capivasertib, or a pharmaceutically acceptable salt thereof, is administered once daily under an intermittent dosing schedule at a dosage from about 275 mg to about 675 mg. In embodiments, capivasertib, or a pharmaceutically acceptable salt thereof, is administered once daily under an intermittent dosing schedule at a dosage from about 300 mg to about 650 mg.

In embodiments, capivasertib, or a pharmaceutically acceptable salt thereof, is administered twice daily (BID) under an intermittent dosing schedule. In embodiments, capivasertib, or a pharmaceutically acceptable salt thereof, is administered under an intermittent dosing schedule at a dosage from about 100 mg to about 800 mg twice daily. In embodiments, capivasertib, or a pharmaceutically acceptable salt thereof, is administered under an intermittent dosing schedule at a dosage from about 150 mg to about 750 mg twice daily. In embodiments, capivasertib, or a pharmaceutically acceptable salt thereof, is administered under an intermittent dosing schedule at a dosage from about 200 mg to about 700 mg twice daily. In embodiments, capivasertib, or a pharmaceutically acceptable salt thereof, is administered under an intermittent dosing schedule at a dosage from about 225 mg to about 675 mg twice daily. In embodiments, capivasertib, or a pharmaceutically acceptable salt thereof, is administered under an intermittent dosing schedule at a dosage from about 250 mg to about 650 mg twice daily. In embodiments, capivasertib, or a pharmaceutically acceptable salt thereof, is administered under an intermittent dosing schedule at a dosage from about 300 mg to about 625 mg twice daily. In embodiments, capivasertib, or a pharmaceutically acceptable salt thereof, is administered under an intermittent dosing schedule at a dosage from about 200 mg to about 300 mg twice daily. In embodiments, capivasertib, or a pharmaceutically acceptable salt thereof, is administered under an intermittent dosing schedule at a dosage from about 300 mg to about 400 mg twice daily. In embodiments, capivasertib, or a pharmaceutically acceptable salt thereof, is administered under an intermittent dosing schedule at a dosage from about 400 mg to about 500 mg twice daily. In embodiments, capivasertib, or a pharmaceutically acceptable salt thereof, is administered under an intermittent dosing schedule at a dosage from about 500 mg to about 600 mg twice daily. In embodiments, capivasertib, or a pharmaceutically acceptable salt thereof, is administered under an intermittent dosing schedule at a dosage from about 600 mg to about 700 mg twice daily. In embodiments, capivasertib, or a pharmaceutically acceptable salt thereof, is administered under an intermittent dosing schedule at a dosage from about 700 mg to about 800 mg twice daily. In embodiments, capivasertib, or a pharmaceutically acceptable salt thereof, is administered under an intermittent dosing schedule at a dosage of about 160 mg twice daily. In embodiments, capivasertib, or a pharmaceutically acceptable salt thereof, is administered under an intermittent dosing schedule at a dosage of about 200 mg twice daily. In embodiments, capivasertib, or a pharmaceutically acceptable salt thereof, is administered under an intermittent dosing schedule at a dosage of about 240 mg twice daily. In embodiments, capivasertib, or a pharmaceutically acceptable salt thereof, is administered under an intermittent dosing schedule at a dosage of about 280 mg twice daily. In embodiments, capivasertib, or a pharmaceutically acceptable salt thereof, is administered under an intermittent dosing schedule at a dosage of about 320 mg twice daily. In embodiments, capivasertib, or a pharmaceutically acceptable salt thereof, is administered under an intermittent dosing schedule at a dosage of about 360 mg twice daily. In embodiments, capivasertib, or a pharmaceutically acceptable salt thereof, is administered under an intermittent dosing schedule at a dosage of about 400 mg twice daily. In embodiments, capivasertib, or a pharmaceutically acceptable salt thereof, is administered under an intermittent dosing schedule at a dosage of about 440 mg twice daily. In embodiments, capivasertib, or a pharmaceutically acceptable salt thereof, is administered under an intermittent dosing schedule at a dosage of about 480 mg twice daily. In embodiments, capivasertib, or a pharmaceutically acceptable salt thereof, is administered under an intermittent dosing schedule at a dosage of about 520 mg twice daily. In embodiments, capivasertib, or a pharmaceutically acceptable salt thereof, is administered under an intermittent dosing schedule at a dosage of about 580 mg twice daily. In embodiments, capivasertib, or a pharmaceutically acceptable salt thereof, is administered under an intermittent dosing schedule at a dosage of about 600 mg twice daily. In embodiments, capivasertib, or a pharmaceutically acceptable salt thereof, is administered under an intermittent dosing schedule at a dosage of about 640 mg twice daily. In embodiments, capivasertib is administered under an intermittent dosing schedule at a dosage of about 680 mg twice daily. In embodiments, capivasertib, or a pharmaceutically acceptable salt thereof, is administered under an intermittent dosing schedule at a dosage of about 720 mg twice daily. In embodiments, capivasertib, or a pharmaceutically acceptable salt thereof, is administered under an intermittent dosing schedule at a dosage of about 760 mg twice daily. In embodiments, capivasertib, or a pharmaceutically acceptable salt thereof, is administered under an intermittent dosing schedule at a dosage of about 800 mg twice daily.

In embodiments, the daily dosage of afuresertib, or a pharmaceutically acceptable salt thereof, is administered in the range of about 1 mg/day to about 1,000 mg/day; suitably, the amount will be selected from about 40 mg to about 120 mg. Accordingly, the amount of afuresertib, or a pharmaceutically acceptable salt thereof, administered as part of the combination according to the present invention may be an amount selected from about 40 mg to about 160 mg. For example, the amount of Compound A, or a pharmaceutically acceptable salt thereof, administered as part of the combination according to the present invention can be 40 mg, 80 mg, 120 mg, or 160 mg.

In embodiments, the daily dosage of afuresertib is administered in the range of about 1 mg/day to about 1,000 mg/day; suitably, the amount will be selected from about 40 mg to about 120 mg. Accordingly, the amount of afuresertib administered as part of the combination according to the present invention may be an amount selected from about 40 mg to about 160 mg. For example, the amount of Compound A administered as part of the combination according to the present invention can be 40 mg, 80 mg, 120 mg, or 160 mg.

The selected amount of afuresertib, or a pharmaceutically acceptable salt thereof, is administered daily, wherein the amount of afuresertib, or a pharmaceutically acceptable salt thereof, may be administered once or multiple times daily. Suitably, the selected amount of afuresertib, or a pharmaceutically acceptable salt thereof, is administered from 1 to 4 times a day. For example, afuresertib, or a pharmaceutically acceptable salt thereof, is administered at an amount of 4×40 mg administered once a day.

The selected amount of afuresertib is administered daily, wherein the amount of afuresertib may be administered once or multiple times daily. Suitably, the selected amount of afuresertib is administered from 1 to 4 times a day. For example, afuresertib is administered at an amount of 4×40 mg administered once a day.

Repetition of the administration or dosing regimens may be conducted as necessary to achieve the desired reduction or diminution of cancer cells. A “continuous dosing schedule,” as used herein, is an administration or dosing regimen without dose interruptions, e.g., without days off treatment. Repetition of 28-day treatment cycles without dose interruptions between the treatment cycles is an example of a continuous dosing schedule. In embodiments, the compounds of the combination of the present invention can be administered in a continuous dosing schedule. In embodiments, the compounds of the combination disclosed herein can be administered concurrently in a continuous dosing schedule.

In embodiments, Compound A, or a pharmaceutically acceptable salt thereof, is administered once daily to comprise a complete cycle of 28-days. Repetition of 28-day treatment cycles is continued during treatment in accordance with the methods and uses of the present disclosure.

The standard recommended dosing regimen, which includes the standard dosing schedule, for palbociclib, or a pharmaceutically acceptable salt thereof, is administration once daily for 21 consecutive days followed by 7 days off treatment to comprise a complete cycle of 28-days. Repetition of the 28-day cycles is continued during treatment with the combination of the present invention.

The standard recommended dosing regimen, which includes the standard dosing schedule, for capivasertib, or a pharmaceutically acceptable salt thereof, is administration orally twice daily for 4 consecutive days followed by 3 days off treatment each week to comprise a complete cycle of 28-days. Repetition of the 28-day cycles is continued during treatment with the combination of the present invention.

Also disclosed herein are kits comprising the therapeutic agents of the combination of the present disclosure and written instructions for administration of the therapeutic agents. In embodiments, the written instructions elaborate and qualify the modes of administration of the therapeutic agents, for example, for simultaneous or sequential administration of the therapeutic agents of the present disclosure. In embodiments, the written instructions elaborate and qualify the modes of administration of the therapeutic agents, for example, by specifying the days of administration for each of the therapeutic agents during a 28-day treatment cycle.

### Methods of Treatment

In embodiments, provided herein are methods for treating cancer in a subject comprising administering to the subject an effective amount of Compound A as described herein in combination with an amount of an Akt inhibitor.

The term “combination,” as used herein, unless otherwise indicated, refers to the use of Compound A with one or more therapeutic agents, wherein Compound A and the one or more therapeutic agents are administered intermittently, concurrently, or sequentially, according to the same or different route of administration and according to the same or different dosage schedules.

The term “sequential” or “sequentially” refers to the administration of each therapeutic agent of the combination of the invention, either alone or in a medicament, one after the other, wherein each therapeutic agent can be administered in any order. Sequential administration is particularly useful when the therapeutic agents in the combination are in different dosage forms, for example, one agent is a tablet and another agent is a sterile liquid, and/or are administered according to different dosing schedules, for example, one agent is administered daily, and the second agent is administered less frequently such as weekly.

The term “concurrently” refers to the administration of each therapeutic agent in the of the invention, either alone or in separate medicaments, wherein the second therapeutic agent is administered immediately after the first therapeutic agent, but that the therapeutic agents can be administered in any order. In a preferred embodiment the therapeutic agents are administered concurrently.

The term “intermittent” or “intermittently” refers to the administration of each therapeutic agent in the combination of the invention, either alone or in separate medicaments, wherein one of the therapeutic agents is stopped and then restarted. Intermittent dosing may utilize regular “drug holidays” in which a patient is not exposed to drug.

The term “locally advanced,” as used herein, as it relates to cancer, may or may not be treated with curative intent. For example, locally advanced breast cancer (LABC) is defined by the U.S. National Comprehensive Cancer Network as a subset of breast cancer characterized by the most advanced breast tumors in the absence of distant metastasis, wherein the tumors are more than 5 cm in size with regional lymphadenopathy; tumors of any size with direct extension to the chest wall or skin, or both (including ulcer or satellite nodules), regardless of regional lymphadenopathy; presence of regional lymphadenopathy (clinically fixed or matted axillary lymph nodes, or any of infraclavicular, supraclavicular, or internal mammary lymphadenopathy) regardless of tumor stage. (Garg et al. Curr Oncol. 2015 October; 22 (5): e409-10; National Comprehensive Cancer Network NCCN Clinical Practice Guidelines in Oncology: Breast Cancer. Fort Washington, PA: NCCN; 2015. Ver. 2.2015.)

The term “metastatic” as used herein, as it relates to cancer, cannot be treated with curative intent. For example, metastatic breast cancer refers to breast cancer that has spread beyond the breast and nearby lymph nodes to other parts of the body, e.g., bones, liver, lungs, or brain. (www.cancer.org/cancer/breast-cancer.)

Those skilled in the art will be able to recognize and diagnose locally advanced and metastatic cancer in a patient or subject.

For convenience, certain well-known abbreviations may be used herein, including: castration resistant prostate cancer (CRPC), estrogen receptor positive (ER+), human epidermal growth factor receptor 2 negative (HER2−), hormone receptor (HR), human epidermal growth factor receptor 2 positive (HER2+), non-small cell lung cancer (NSCLC), and progesterone receptor (PR).

In embodiments, the cancer is selected from lung cancer, mesothelioma, bone cancer, pancreatic cancer, skin cancer, cancer of the head or neck, cutaneous or intraocular melanoma, uterine cancer, ovarian cancer, rectal cancer, cancer of the anal region, stomach cancer, hepatic carcinoma, colon cancer, breast cancer, uterine cancer, carcinoma of the fallopian tubes, carcinoma of the endometrium, carcinoma of the cervix, carcinoma of the vagina, carcinoma of the vulva, Hodgkin's disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, hematology malignancy, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, glioblastoma, brain stem glioma, pituitary adenoma, head and neck cancer, and combinations of two or more of the foregoing cancers.

Also disclosed herein are methods of treating cancer in a subject. In embodiments, the methods comprise treating cancer in a subject comprising administering to the subject an amount of the compounds described herein that are effective in treating the cancer.

In embodiments, the cancer is breast cancer, lung cancer, colon cancer, brain cancer, head and neck cancer, prostate cancer, stomach cancer, pancreatic cancer, ovarian cancer, melanoma cancer, endocrine cancer, uterine cancer, testicular cancer, or bladder cancer.

In embodiments, the cancer is breast cancer, lung cancer, prostate cancer, pancreatic cancer, or ovarian cancer.

In embodiments, the cancer is breast cancer, lung cancer, or prostate cancer.

In embodiments, the cancer is breast cancer.

In embodiments, the breast cancer is metastatic breast cancer.

In embodiments, the breast cancer is locally advanced breast cancer.

In embodiments, the breast cancer is HR+ breast cancer.

In embodiments, the HR+ breast cancer is PR+ and/or ER+ breast cancer.

In some embodiments, the breast cancer is PR+ breast cancer.

In embodiments, the breast cancer is ER+ breast cancer.

In embodiments, the breast cancer is ER+ HER2− breast cancer.

In embodiments, the breast cancer is ER+ HER2+ breast cancer.

In embodiments, the breast cancer is locally advanced or metastatic ER+ breast cancer.

In embodiments, the breast cancer is locally advanced or metastatic ER+ HER2− breast cancer.

In embodiments, the breast cancer is locally advanced or metastatic ER+ HER2+ breast cancer.

In embodiments, the breast cancer is metastatic, ER+, HER2− breast cancer.

In embodiments, the breast cancer is metastatic, ER+, HER2− breast cancer that is also locally advanced.

In embodiments, the lung cancer is non-small cell lung cancer.

In embodiments, the lung cancer is locally advanced or metastatic non-small cell lung cancer.

In embodiments, the prostate cancer is CRPC.

In embodiments, the prostate cancer is locally advanced or metastatic CRPC.

Also disclosed herein are methods of treating solid tumors in a subject. In embodiments, disclosed herein are methods of treating solid tumors in a subject comprising administering to the subject an amount of the compounds described herein that are effective in treating the solid tumor.

In embodiments, the solid tumor is breast cancer, lung cancer, colon cancer, brain cancer, head and neck cancer, prostate cancer, stomach cancer, pancreatic cancer, ovarian cancer, melanoma cancer, endocrine cancer, uterine cancer, testicular cancer, or bladder cancer.

In embodiments, the solid tumor is breast cancer, lung cancer, prostate cancer, pancreatic cancer, or ovarian cancer.

In embodiments, the solid tumor is breast cancer, lung cancer, or prostate cancer.

In embodiments, the solid tumor is breast cancer. For example, in certain embodiments the breast cancer is HR+ breast cancer. In other embodiments, the HR+ breast cancer is PR+ and/or ER+ breast cancer ER+ breast cancer.

In embodiments, the solid tumor is breast cancer. For example, in certain embodiments, the breast cancer is ER+ HER2− breast cancer.

In embodiments, the solid tumor is breast cancer. For example, in certain embodiments, the breast cancer is ER+ HER2+ breast cancer.

In embodiments, the solid tumor is breast cancer. For example, in certain embodiments, the breast cancer is locally advanced or metastatic ER+ HER2− breast cancer.

In embodiments, the solid tumor is breast cancer. For example, in certain embodiments, the breast cancer is locally advanced or metastatic ER+ HER2+ breast cancer.

In embodiments, the solid tumor is lung cancer. For example, in certain embodiments, the lung cancer is non-small cell lung cancer.

In embodiments, the solid tumor is lung cancer. For example, in certain embodiments, the lung cancer is locally advanced or metastatic non-small cell lung cancer.

In embodiments, the solid tumor is prostate cancer. For example, in certain embodiments, the prostate cancer is CRPC.

In embodiments, the solid tumor is prostate cancer. For example, in certain embodiments, the prostate cancer is locally advanced or metastatic castration resistant prostate cancer.

Also disclosed herein are methods of treating hematologic tumors in a subject. In certain embodiments, the method comprises treating hematologic tumors in a subject comprising administering to the subject an amount of the compounds described herein that is effective in treating the hematologic tumor.

In embodiments, the hematologic tumor is leukemia, lymphoma, or multiple myeloma.

In embodiments, the hematologic tumor is leukemia or lymphoma.

Also disclosed herein are methods of treating cancer in a subject with locally advanced or metastatic ER+ HER2− breast cancer, CRPC, or NSCLC whose disease progressed on or is intolerant to standard therapy.

Also disclosed herein are methods of treating cancer in a subject with locally advanced or metastatic ER+ HER2− breast cancer, CRPC, or NSCLC whose disease progressed on or is intolerant to standard therapy.

Also disclosed herein are methods of treating cancer in a subject with locally advanced or metastatic 2L+ ER+ HER2 breast cancer who have received prior hormonal/endocrine therapy and chemotherapy in the locally advanced/metastatic setting. In embodiments, the method comprises administering Compound A in combination with an Akt inhibitor to the subject.

Also disclosed herein are therapeutic combinations comprising:
(i) Compound A, or a pharmaceutically acceptable salt thereof; and
(ii) an Akt inhibitor;
for simultaneous, separate, or sequential use in a method of treating cancer; wherein the method comprises administering Compound A, or a pharmaceutically acceptable salt thereof.

Also disclosed herein are therapeutic combinations comprising:
(i) Compound A; and
(ii) an Akt inhibitor;
for simultaneous, separate, or sequential use in a method of treating cancer; wherein the method comprises administering Compound A, or a pharmaceutically acceptable salt thereof.

Also disclosed herein is Compound A, or a pharmaceutically acceptable salt thereof, for use in a method of treating cancer, wherein the method comprises administering Compound A, or a pharmaceutically acceptable salt thereof, and wherein the method further comprises administering an Akt inhibitor.

Also disclosed herein is Compound A, for use in a method of treating cancer, wherein the method comprises administering Compound A, or a pharmaceutically acceptable salt thereof, and wherein the method further comprises administering an Akt inhibitor.

Also disclosed herein is an Akt inhibitor for use in a method of treating cancer, wherein the method further comprises administering Compound A, or a pharmaceutically acceptable salt thereof.

Also disclosed herein is an Akt inhibitor for use in a method of treating cancer, wherein the method further comprises administering Compound A.

In embodiments, the method comprises administering a daily dose of about 200 mg of Compound A, or a pharmaceutically acceptable salt thereof. In embodiments, the method comprises administering a daily dose of about 200 mg of Compound A.

In embodiments, the method comprises administering a daily dose of 200 mg of Compound A, or a pharmaceutically acceptable salt thereof. In embodiments, the method comprises administering a daily dose of 200 mg of Compound A.

In embodiments, the method comprises administering a daily dose of about 100 mg of Compound A, or a pharmaceutically acceptable salt thereof. In embodiments, the method comprises administering a daily dose of about 100 mg of Compound A.

In embodiments, the method comprises administering a daily dose of 100 mg of Compound A, or a pharmaceutically acceptable salt thereof. In embodiments, the method comprises administering a daily dose of 100 mg of Compound A.

### EXEMPLIFICATION

In order that this invention may be better understood, the following examples are set forth. These examples are for purposes of illustration only and are not to be construed as limiting the scope of the invention in any manner.

Example 1: Enhanced Efficacy of Compound a in Combination with the Akt Inhibitors Capivasertib and Afuresertib in ER+ Breast Cancer Models

### Methods

### Live-Cell Imaging Proliferation Assay

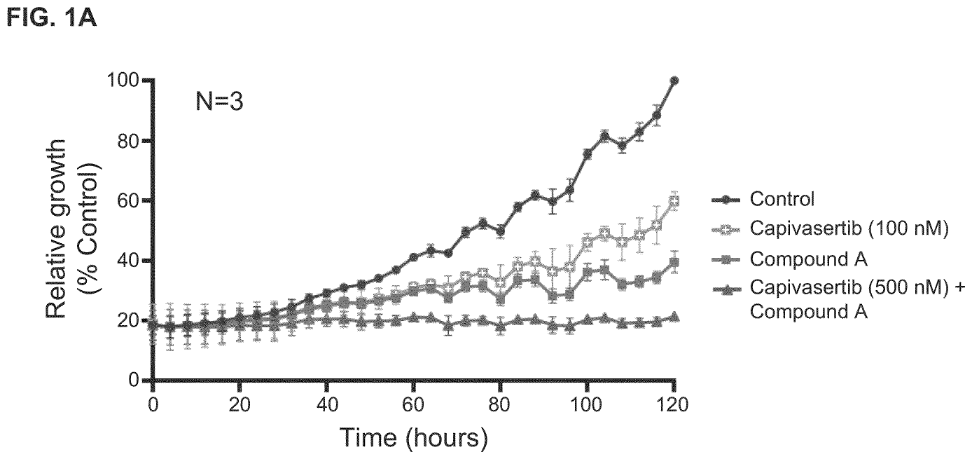

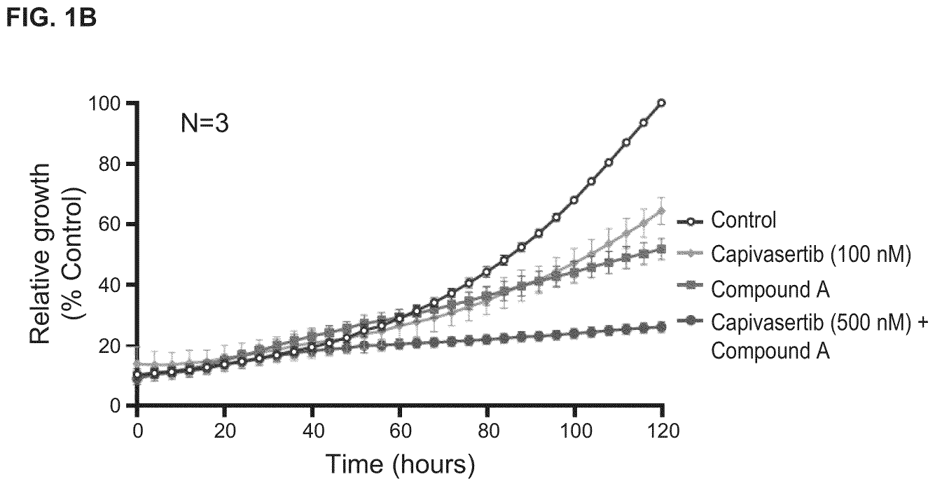

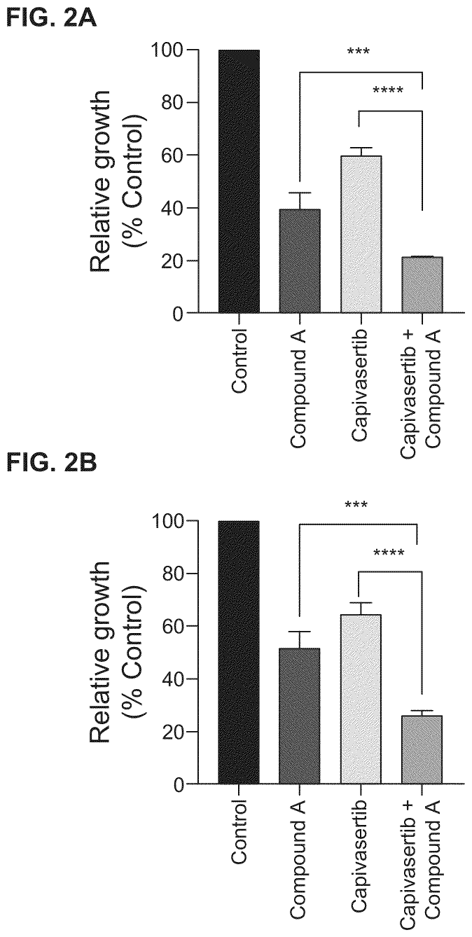

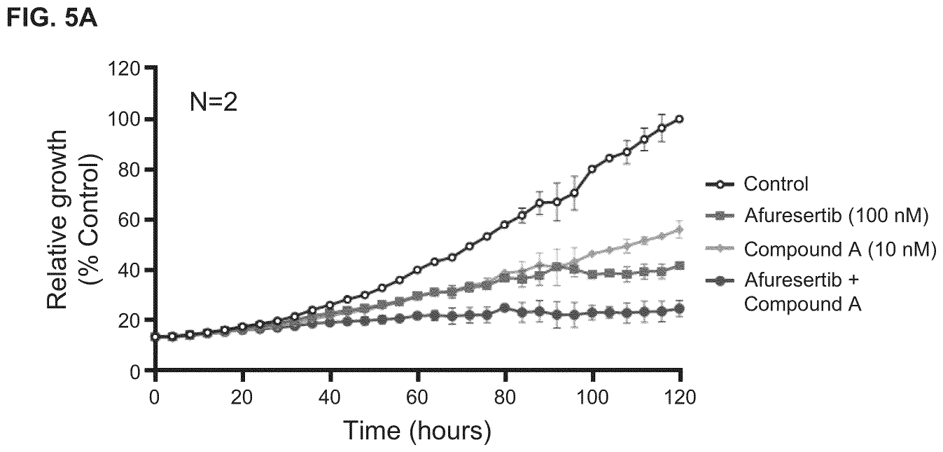

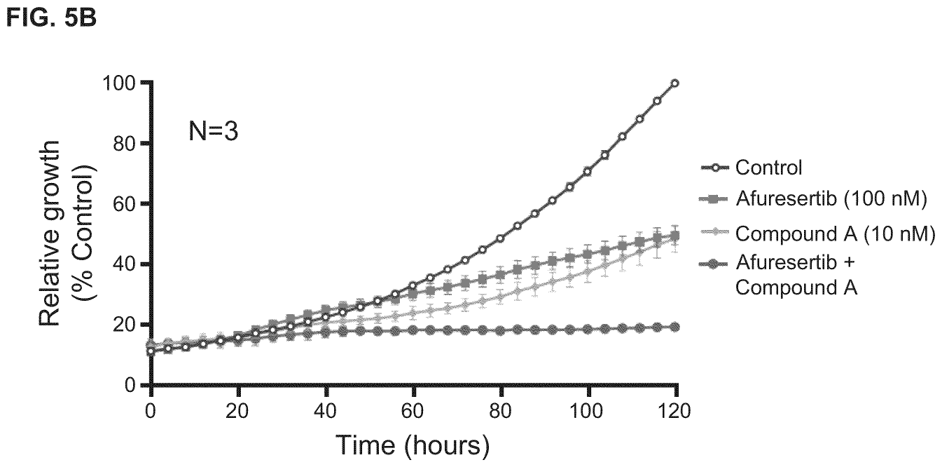

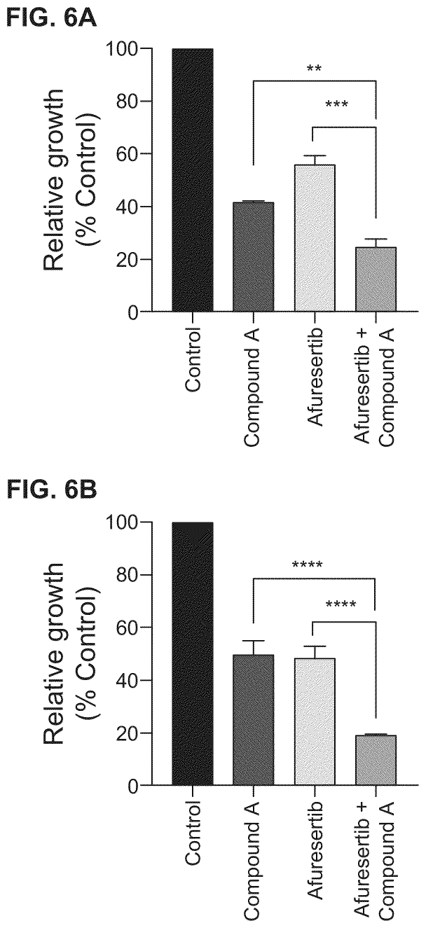

MCF7 or T47D cells were seeded in 6 well plates and treated with the indicated concentrations of compounds. The plate was then placed in the Incucyte® S3 Live-Cell Analysis System and images were acquired every 4 hours for a total of 5 days. Data were analyzed using the Incucyte® Software v2020C, which quantified cell surface area coverage as confluence values. Relative growth was calculated for all timepoints for all growth conditions relative to the confluence value observed for the control at 120 hours (FIGS. 1A, 1B, 5A, and 5B). Graphing and statistical analyses were performed using Graphpad Prism (GraphPad Software).

### Dose-Response Matrix Assay

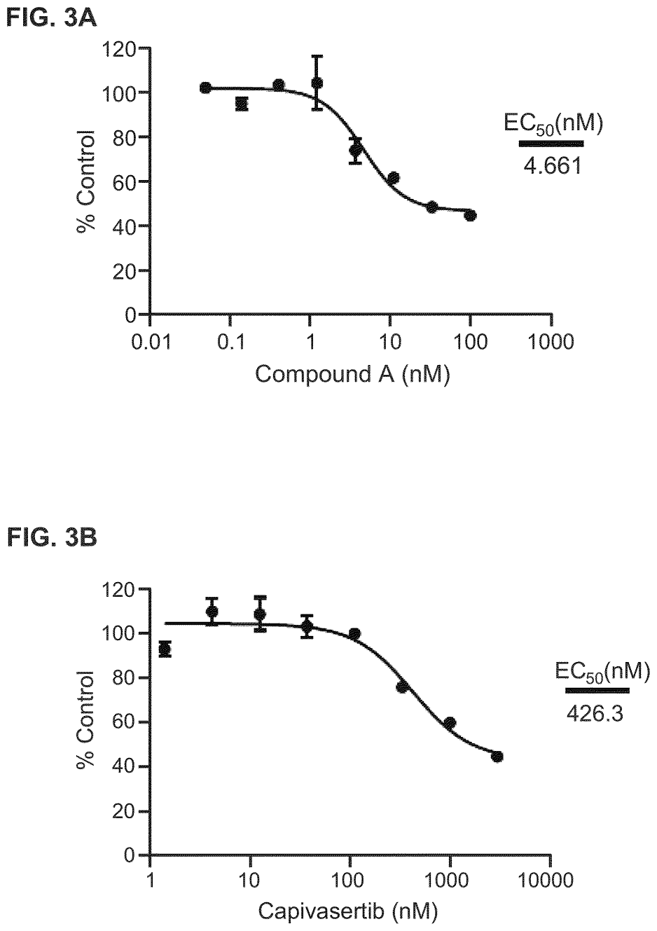

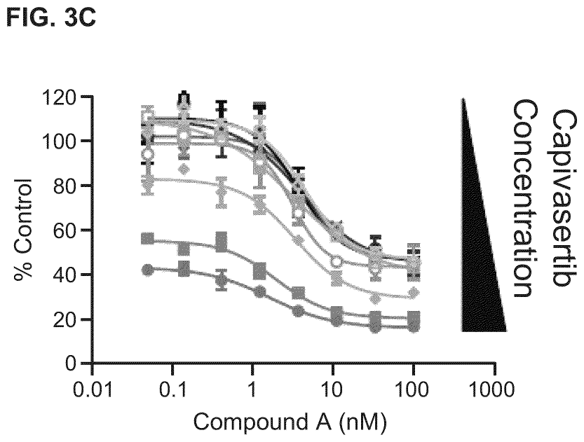

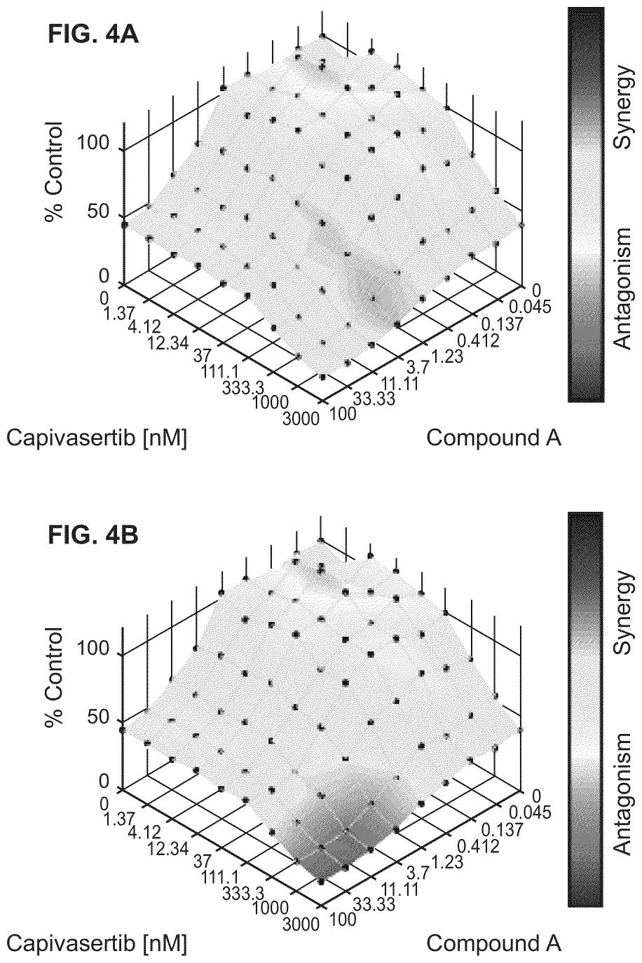

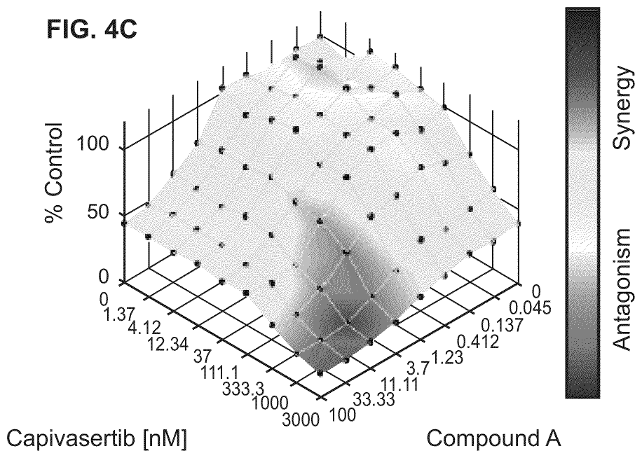

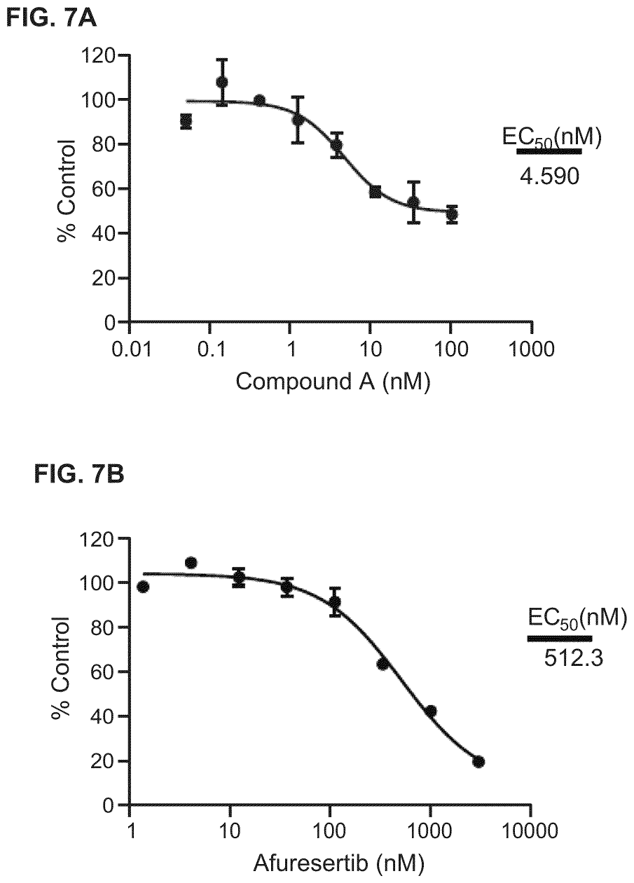

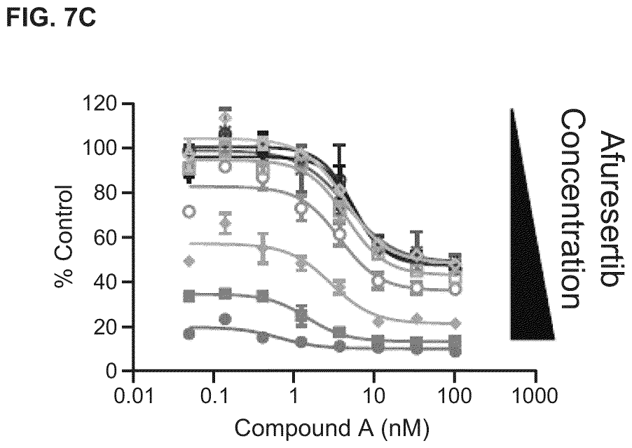

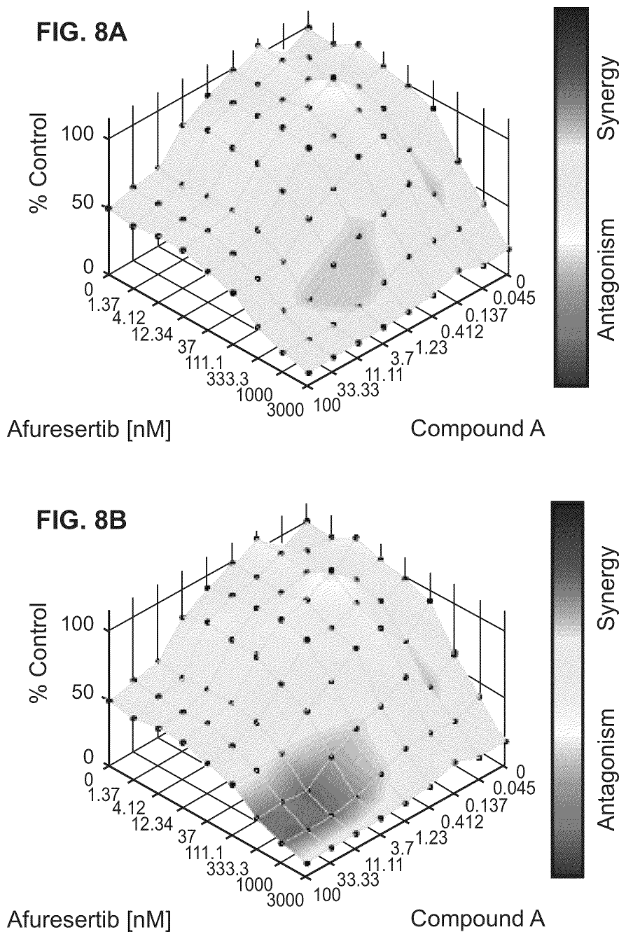

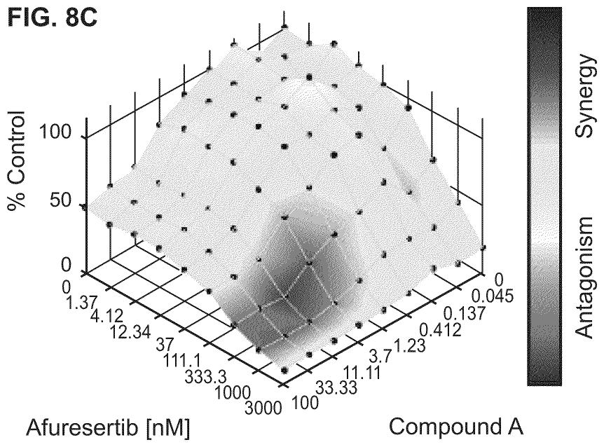

Cells were seeded at 2×10³ cells in 200 μl of media per well in 96 well plates and incubated overnight at 37 °C. Compound A and capivasertib or afuresertib concentrations curves were started at 100 nM for an 8-point concentration curve ranging from 100 to 0.046 nM (FIGS. 3A-3C and 7A-7C). At Day 5 cell viability was measured using Cell-Titer Glo and CTG data were analyzed with the Combenefit Software (5). The results from these assays show evidence of synergy between Compound A and either capivasertib or afuresertib.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

The T methods of the disclosure have been described herein by reference to certain preferred embodiments. However, as particular variations thereon will become apparent to those skilled in the art, based on the disclosure set forth herein, the disclosure is not to be considered as limited thereto.

Unless otherwise defined, all technical and scientific terms used herein have the same meaning as commonly understood by one of ordinary skill in the art to which this disclosure belongs. In the specification and claims, the singular forms also include the plural unless the context clearly dictates otherwise.

It is to be understood that at least some of the descriptions of the disclosure have been simplified to focus on elements that are relevant for a clear understanding of the disclosure, while eliminating, for purposes of clarity, other elements that those of ordinary skill in the art will appreciate may also comprise a portion of the disclosure. However, because such elements are well known in the art, and because they do not necessarily facilitate a better understanding of the disclosure, a description of such elements is not provided herein.

Further, to the extent that a method does not rely on the particular order of steps set forth herein, the particular order of the steps recited in a claim should not be construed as a limitation on that claim.

All patents, patent applications, references and publications cited herein are fully and completely incorporated by reference as if set forth in their entirety. Such documents are not admitted to be prior art to the present disclosure.

We claim:

1. A method for treating cancer comprising administering to a subject Compound A: 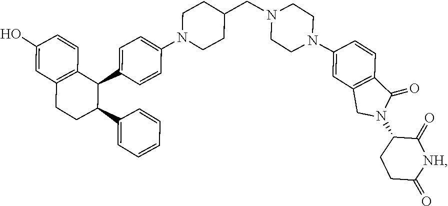 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]
or a pharmaceutically acceptable salt thereof, in combination with an Akt inhibitor.

2. The method of claim 1, wherein a daily dose of Compound A, or a pharmaceutically acceptable salt thereof, is administered to the subject.

3. The method of claim 2, wherein the daily dose of Compound A, or a pharmaceutically acceptable salt thereof, is about 200 mg.

4. The method of any one of claims 1 to 3, wherein the Akt inhibitor is administered concurrently or sequentially.

5. The method of any one of claims 1 to 4, wherein the daily dose of Compound A, or a pharmaceutically acceptable salt thereof, is administered once per day (QD).

6. The method of any one of claims 1 to 5, wherein the daily dose of Compound A, or a pharmaceutically acceptable salt thereof, is administered orally to the subject.

7. The method of any one of claims 1 to 6, wherein the subject is in a fed state.

8. The method of any one of claims 1 to 7, wherein the Akt inhibitor is capivasertib, afuresertib, ipatasertib, MK-2206, miransertib, or a pharmaceutically acceptable salt thereof.

9. The method of claim 8, wherein the Akt inhibitor is capivasertib or afuresertib, or a pharmaceutically acceptable salt thereof.

10. The method of claim 8 or 9, wherein the Akt inhibitor is capivasertib, or a pharmaceutically acceptable salt thereof.

11. The method of claim 8, wherein the Akt inhibitor is afuresertib, or a pharmaceutically acceptable salt thereof.

12. The method of any one of claims 9 to 11, wherein Compound A, or a pharmaceutically acceptable salt thereof, is administered daily in 28-day cycles and the Akt inhibitor is administered orally once a day for 21 days followed by 7 days off treatment for each 28-day cycle.

13. The method of claim 12, wherein the Akt inhibitor is capivasertib, or a pharmaceutically acceptable salt thereof.

14. The method of claim 12, wherein the Akt inhibitor is afuresertib, or a pharmaceutically acceptable salt thereof.

15. The method of any one of claims 1 to 14, wherein the cancer is breast cancer, lung cancer, colon cancer, brain cancer, head and neck cancer, prostate cancer, stomach cancer, pancreatic cancer, ovarian cancer, melanoma, endocrine cancer, uterine cancer, testicular cancer, or bladder cancer.

16. The method of claim 15, wherein the cancer is breast cancer, lung cancer, prostate cancer, pancreatic cancer, or ovarian cancer.

17. The method of claim 16, wherein the cancer is breast cancer, lung cancer, or prostate cancer.

18. The method of claim 17, wherein the cancer is breast cancer.

19. The method of claim 18, wherein the breast cancer is metastatic or locally advanced.

20. The method of claim 18 or 19, wherein the breast cancer is estrogen receptor positive (ER+) breast cancer.

21. The method of claim 20, wherein the estrogen receptor positive (ER+) breast cancer is human epidermal growth factor receptor 2 negative (HER2−).

22. The method of any one of claims 1 to 21, wherein the subject is human.
